(19) United States
(12) Patent Application Publication
(10) Pub. No.: US 2009/0005345 A1
(43) Pub. Date: Jan. 1, 2009

(54) Detoxification of Shellfish

(75) Inventors: Geir Kroken, Vollen (NO); Jan Per Loken, Nordfjordeid (NO); Bjorn Myklebust, Nordfjordeid (NO)

Correspondence Address: VOLPE AND KOENIG, P.C., UNITED PLAZA, SUITE 1600, 30 SOUTH 17TH STREET, PHILADELPHIA, PA, 19103, US

(73) Assignee: FJORD TECHNOLOGY AS, Nordfjordeid (NO)

(21) Appl. No.: 11/908,945

(22) PCT Filed: Mar. 17, 2006
(86) PCT No.: PCT/NO2006/000101

§ 371 Date: Feb. 8, 2008

(30) Foreign Application Priority Data
Mar. 18, 2005 (NO) 20051457

(51) Int. Cl. A61K 31/685; A61K 31/66

(52) U.S. Cl. 514/78; 514/75

(57) ABSTRACT

The present invention relates to the detoxification of bivalves and other shellfish. More particularly, the invention relates to a feed composition and a method for detoxifying shellfish, and the use of a surface-active agent for the detoxification of bivalves and other shellfish.

### FIELD OF THE INVENTION

The present invention relates to the detoxification of bivalves and other shellfish. More particularly, the invention relates to a feed composition and a method for detoxifying shellfish, and to the use of a surface-active agent for the detoxification of bivalves and other shellfish.

### BACKGROUND OF THE INVENTION

In the Scandinavian bivalve industry, algal toxins in bivalves are a major problem for the industry. In Norway, 913 tonnes of mussels were produced in 2001 (Source: Norwegian Directorate of Fisheries), and the main obstruction to further growth in production is algal toxins (Havbruksrapport 2003, Norwegian Institute of Marine Research). Algal toxins can be split into two main groups: water-soluble toxins and fat-soluble toxins. It is the fat-soluble toxins that pose a problem for the bivalve industry. And it is because of the fat-soluble algal toxin Diarrhetic Shellfish Poisoning toxin (DSP toxin or DST), in particular, that bivalves cannot be harvested without a substantial loss in earnings.

Bivalves are cultured by first setting out larvae collectors in the sea, i.e., many kilometres of ropework to which the pelagic bivalve larvae becomes attached. There, they grow in size on the food supply that is found naturally in the surrounding water. When they have become large enough, they are harvested and sold. Because of their special food intake system, which is a filtering system, bivalves make best use of nutrients in the form of particles having a particle size of between 2 and 300 μm.

Bivalves thrive extremely well in brackish water and are therefore often cultured in the arms of fjords. Here, they avoid nutritional competition with the many pure salt water organisms. This means that the bivalves grow quickly, have a large degree of fullness (food content) and are of a very high quality. The lack of competition also means that a considerably greater yield per area unit is achieved in these areas than in the littoral areas. The drawback is that it is in these brackish water areas that the problem of algal blooms of DSP-producing species is greatest.

The DSP toxin consists of the fat-soluble compounds okadaic acid and dinophysitoxin-1, and derivatives thereof. These substances are produced by the algal species Dinophysis, among others, and give symptoms such as abdominal pain, headache and diarrhea in humans. The bivalve producers have hitherto been referred to so-called natural detoxification to solve this problem, i.e., that the toxic bivalves remain in the sea until the toxin level in them is reduced to an acceptable level. This may take a short or long period, after which the bivalves become toxic again. Consequently, there is a great need for efficient detoxification methods which provide predictability for the bivalve producers and safety for the consumer.

Several different methods have been proposed in order to solve the aforementioned problem. All are based on a natural detoxification where the bivalves themselves break down the algal toxin within them in an environment that does not contain algal toxin. Known methods are:

1) The submersion of bivalves to deeper layers of water where the occurrence of toxic algae is smaller. Bøhle et al. (Flødevigen meldinger 2, Norwegian Institute of Marine Research, 1987) have reported that DSP-toxin containing bivalves lowered to an ocean depth of 30 metres became toxin-free after almost five months when measured using the Mouse assay. The condition of these bivalves was greatly reduced as a result of reduced temperature and a lower supply of nutrition, and therefore this is not a solution for the commercial production of shellfish.

2) Moving bivalve production to toxin-free areas between inner and outer fjord regions depending on the time of year in order to avoid areas containing DSP-producing algae. This results in unpredictable production as it is not possible to safeguard against algae that produce other toxins.

3) Feeding bivalves with toxin-free algae in water-based facilities on land. Marcaillou-LeBaut et al. (Smayda & Shimizu (ed.), Elsevier 1993) have reported that bivalves containing okadaic acid (DSP toxin) fed with the toxin-free alga Tetraselmis suesica were harmless after 30 days. In addition to the fact that the production of algae for feed in costly and thus a challenge in relation to a profitable detoxification of bivalves, the feed method has a further limitation which relates to how much toxin there is in the bivalve to begin with. There should not be more toxin in the bivalves than can be reduced to an acceptable level in the course of three weeks. At a toxin concentration of 160 μg/kg, shellfish are reckoned to be toxin-free. Feeding with toxin-free algae under optimal conditions results in an effective half-life of 18-26 days. On this basis, there cannot be more than 280-360 μg of DSP per kg at the outset in order to be able to detoxify the bivalves within a reasonable time. The toxin level is very often higher in sea areas which otherwise have the best quality. This method is therefore not commercially interesting.

As mentioned, the challenges of water-soluble toxins do not pose a large problem. The water-soluble toxins can be removed under controlled conditions in land-based facilities. The toxins are removed after a short time without any treatment other than being placed in purified seawater. It is the fat-soluble or lipophilic toxins that represent a major challenge for the bivalve industry.

Algal toxins are a problem not only in Norway and Scandinavia, but also in many countries with bivalve production. An efficient way of detoxifying bivalves would therefore be of great financial significance. It has been assumed that it is necessary to have a certain percentage of algae in synthetic bivalve feed, but as already mentioned, algae production is costly. On the other hand, a problem of algae-free feed is that essential elements in the feed are inaccessible to the digestive system of bivalves. The feed particles pass out undigested in the faeces or in the form of so-called pseudofaeces. Feeding with, for example, whole bacteria and yeast cells has therefore been found to be useless in connection with detoxification.

There is therefore still a need for new agents and methods for detoxifying bivalves and other shellfish.

### DESCRIPTION OF THE INVENTION

The present invention relates to the detoxification of bivalves and other shellfish. The present inventors have found surprisingly that bivalves and other shellfish can be detoxified by moving them to a land-based facility once they have reached a satisfactory size and supplying them with an emulsifier which binds the lipophilic toxins and passes them out via the faeces. This is in contrast to previously known methods which are based on a slow breakdown of the algal toxin in the bivalve.

Detoxification can be effected by adding the agent to the water in the water-based facility, or by adding it as feed.

The inventors have shown that it is financially viable to remove DSP toxin using a synthetic feed containing protein and lipids, including considerable amounts of surface-active phospholipids. This has been successful in particular because of the special digestive system of bivalves.

Fat-soluble algal toxins are accumulated in the digestive gland (hepatopancreas) in shellfish. The hepatopancreas is a gland organ which is not found in mammals and can be described as a combination of the liver and the pancreas. In bivalves, the hepatopancreas lies around the stomach and is also connected thereto by ducts. From certain areas in the stomach, streams of feed particles are formed which are directed towards these digestive ducts, which means that feed comes into close contact with the algal toxin containing cells in the hepatopancreas.

This special digestive system is made use of by the present invention and means that the toxin level in bivalves can be reduced by supplying the stomach and the hepatopancreas with emulsifiers which are surface-active compounds. Examples of such compounds are phospholipids. This characteristic digestive system of bivalves means that the surface-active compounds reach the lipophilic toxins that have accumulated in the hepatopancreas. The toxins are dissolved in the digestive fluid and are returned to the stomach/intestine and the bivalve rids itself of the toxins via its faeces.

One object of the present invention is to provide a feed composition containing an emulsifier.

Another object of the present invention is to provide a method for detoxifying bivalves and other shellfish.

Yet another object of the present invention is the use of an emulsifier for detoxifying bivalves and other shellfish.

These and other objects are achieved by means of the present invention.

### DETAILED DESCRIPTION OF THE INVENTION

Accordingly, the present invention relates to a feed composition for detoxifying bivalves and other shellfish, characterised in that it comprises an emulsifier. Any emulsifier and other surface-active agents are suitable. Phospholipids, preferably lecithins, are particularly suitable.

The feed is fed to the bivalves after they have been placed in land-based facilities. Without being bound by any particular theory, it is suggested that emulsifiers or other surface-active compounds take up the organic or lipophilic toxins found in bivalves and other shellfish, which results in a rapid and substantial reduction in the toxin level.

The emulsifier can be added as a component of a feed which otherwise consists of standard feed components such as fat or oil, carbohydrates, proteins, vitamins and micronutrients. The purpose of such a feed is primarily to maintain a metabolism and a degree of fullness in the shellfish during the detoxification process. The oil, which becomes emulsified in the prevailing environment with surface-active compounds, may be any oil, preferably fish oil such as cod liver oil, or another oil rich in unsaturated fatty acids. This is expedient in order to be able to replace any loss of such fatty acids during the detoxification process. The feed is preferably in the form of particles having a size of from 2 μm to 300 μm. Particles which are smaller than 2 μm are normally seldom captured as food for bivalves.

The addition of carbohydrates to the feed composition is not necessary for detoxification, but leads to a higher degree of fullness and gives the bivalve meat a more attractive colour. Consequently, the addition of carbohydrates to the feed composition is preferable. The carbohydrate may consist of mono or polysaccharides, but in a preferred embodiment the carbohydrate consists of modified polysaccharides, more specifically modified starch. The modification consists of boiling to obtain so-called “sticky” starch. The starch may be of any origin.

The ratio between the different components of the feed composition according to the invention may vary greatly depending on the availability and price of the raw materials, and it will be within the knowledge of the skilled person to find a functional mixture ratio.

In a preferred embodiment, the invention relates to a feed composition which further comprises a protein source, carbohydrate and/or other standard feed components. There is a need for a supply of nutrients if the bivalves have to remain in the land-based facilities for an extended period of time. One example of a suitable composition is BioProtein® or Basic BioProtein®, (Norferm AS, PO Box 8005, NO-4068 Stavanger, Norway) which consists of 70% protein and 10% fat, most of which, about 8% of the total composition, consists of phospholipids.

An especially preferred embodiment of the feed composition according to the invention comprises

| | |
|---|---|
| Soft roe (emulsion agent) | 0.1 g (3.3%) |
| Cod liver oil (lipid) | 0.1 g (3.3%) |
| Basic BioProtein ® | 2.5 g (83%) |
| Modified starch | 0.3 g (10.4%) |

The percentages are given as percentages by weight based on the total weight of the composition.

The feed composition may be in any form functional for the purpose, for example, a powder, dissolvable pellets, liquid form (concentrated or unconcentrated), slurry, etc. In a preferred embodiment according to the invention, the components specified above are dissolved into a slurry, which is added to the detoxification vessels.

The feeding can be carried out in any sequence, optionally all the feed elements can be mixed together in water to give a feed slurry. In an especially preferred embodiment, the feed composition as described above is used. It is assumed that a skilled person in the field will be capable of optimising the dosage to achieve fastest possible detoxification. As a rule of thumb or a basis, it is however preferable to give 3 grams of feed composition per kg of bivalves. It should also be mentioned that it is very important that overfeeding should not occur as this leads to pseudofaeces.

The present invention further relates to a method for detoxifying mussels and other shellfish by supplying the shellfish with an emulsifier.

In one preferred embodiment, the shellfish placed in land-based facilities are fed with a feed composition according to the invention. The shellfish may, for example, be fed with an oil-in-water emulsion, i.e., phospholipids and emulsified oils or other lipids. The feed given to the shellfish contains carbohydrate and protein as described above in order, inter alia, to maintain their metabolism and their degree of fullness during the detoxification process. The added oil may be of marine origin. In an especially preferred embodiment, this oil is from cod liver. The oil may be emulsified as described above, preferably with phospholipids. Phospholipids from lecithins of vegetable and/or marine origin are especially preferred.

In another preferred embodiment according to the invention, it comprises a method or detoxification process in which the shellfish are washed and rinsed with a medium containing an emulsifier.

Tests carried out by the inventors show that the emulsifier gives a good detoxification effect if it is added alone and in that way acts directly as a tenside. The active ingredients are dissolved in water which the bivalves ingest. Without being bound by any theory, it is believed that the fluid in the digestive ducts which pass deep into the hepatopancreas becomes more lipophilic and dissolves lipids and lipophilic toxins which have accumulated there, and the process works as a washing process. It is preferable to use phospholipids that are rich in unsaturated fatty acids in order to replace any loss thereof during the detoxification process. The phospholipids may be of both marine and vegetable origin and marine or vegetable lecithins are preferred.

The process takes place in tanks on land in which the bivalves live in recycling and aerated seawater. The emulsifier is added to the medium. The medium becomes clouded by the addition in order to then become almost clear. This is defined as the washing process or washing step. The washing process may be carried out continuously or batchwise. After some time, the medium is replaced by purified recycling and aerated seawater. This is defined as the rinsing process or rinsing step. Several washing and rinsing processes follow until the toxin level has been reduced to an acceptable level and the bivalves are defined as toxin-free.

Another embodiment of the invention relates to the use of an emulsifier for detoxifying bivalves and other shellfish. The emulsifier is preferably a phospholipid or lecithin. Commercial bioproteins which also have a relatively high phospholipid content are very suitable.

In a test using a randomly selected amount of emulsifier, excellent results were achieved with a half-life of 1-4 days. This is as opposed to a half-life of about 18-26 days, which is the norm when using algal feed under optimal conditions.

As mentioned above, there has previously been an upper limit of three weeks' treatment time for profitable detoxification in land-based facilities. With a half-life of 4 days obtained by means of the present invention, the toxin concentration in bivalves can be reduced from 10,000 μg/kg to 160 μg/kg in 24 days and the bivalves can be regarded as toxin-free. In this way, it may be profitable to detoxify the otherwise good quality bivalves from, for example, the inner Sognefjord. This has previously been considered impossible. It is actually quite rare to have concentrations higher than 1000 μg/kg. However, situations in which the toxin amount is 300-600 μg/kg are not unusual. On this basis, detoxification will take 4-8 days.

In the present description the focus is on DSP toxins in bivalves. However, it should be made clear that the application comprises all organic or fat-soluble toxins which accumulate in the hepatopancreas or digestive system of bivalves and other shellfish. It thus also comprises organic pollutants or organic environmental poisons. Bivalves filter large amounts of water and only relatively small amounts of environmental poisons are needed before they accumulate in bivalves. This is the case even though bivalves are early in the food chain.

### FIGURES

FIG. 1 is taken from the book Skjell—biologi og dyrkning by Peter Hovgaard, Stein Mortensen and Øivind Strand and shows a basic diagram of the digestive processes that take place in the stomach and digestive gland of a bivalve. On the left, it is shown how the feed comes into the stomach and is distributed to the ducts of the digestive gland. The crystalline style (coloured green) rotates against the stomach wall, releases digestive enzymes and helps to establish turbulence in the stomach. The illustration on the right is an enlargement showing the principles of transport, feed processing, absorption and secretion in a digestive diverticulum. In some areas there are active digestive cells (coloured pink). Mobile haemocytes (coloured blue) pass through the digestive epithelia and contribute to the intracellular digestion. Some cells in the epithelial layers (coloured red) produce and secrete digestive enzymes.

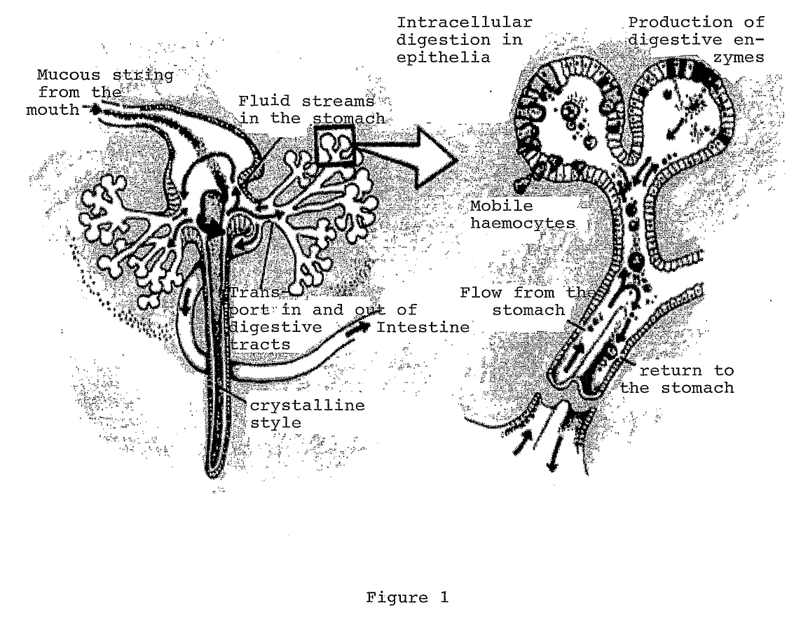

FIG. 2 shows the DSP level as a function of feed time (days) in tests in which bivalves were fed with bioprotein (Test 4) at a water temperature of 10° C. The half-life was calculated to be 8, 10 and 14 days after a detoxification period of respectively 1 week, 2 weeks and about 4 weeks. The limit value of 160 μg/kg DSP is indicated.

FIG. 3 shows the DSP level as a function of feed time (days) in tests in which bivalves were fed with a mixture of bioprotein and emulsified oil (Test 5) at a water temperature of 7.2° C. The calculated half-life was 15, 13 and 13 days after a detoxification period of respectively 10, 20 and 29 days. The limit value of 160 μg/kg DSP is indicated.

### EXAMPLES

### Test Description

Seawater from a depth of 45 metres in the Eidfjord, with a salinity of 33-34 per mille, was pumped up into a reservoir on land. The water was then passed into the test vessels. The bivalves were evenly distributed on the mesh/grating in a layer of about 10 cm in thickness. The vessel diameter is about 70 cm. In these small scale tests importance was given to ensuring that oxygen should not be a limiting factor. That is to say that there is sufficient flow-through of water to obtain approximate oxygen saturation in the outflow. The amount of water through each vessel was about 8 litres per minute.

Feeding was effected automatically batchwise throughout the day. That is to say that the daily consumption of the feed slurry according to Example 1 was filled into a tank once a day. The slurry runs from this tank to the bivalve vessel cascade via a valve. In our tests we chose to feed batchwise once an hour. Such portion feeding is important in order to obtain an even distribution to the whole bivalve population. The feed was added in the course of a few seconds. The volume of the vessel was about 20 litres. With 10 kg of bivalves in the vessel and a feed amount of 3 grams per kg of mussels, the concentration of feed in the vessel was 0.125 g per litre at the feeding time, and this subsequently dropped towards 0 through to the next feeding.

The feed in the feed tank was well aerated to avoid anaerobic conditions. Aquarium pumps were used for this purpose.

The washing and rinsing processes were carried out in the same vessels as the feed tests. The amount of water through the vessels was also 8 litres per minute in these processes. The water was recycled during the washing phase using a vessel of 50 litres of seawater with added lecithin equipped with a pump system. During the rinsing phase, the water was recycled through a similar vessel arrangement of 50 litres of purified seawater.

One vessel was used for each test set-up, including one vessel for a blind test without feeding or washing.

In the case of all the tests, samples were taken immediately prior to the test start and after given time periods. At the start, when taking bivalve samples for analysis, particular importance was given to mixing the bivalves together into a homogeneous mass. This was done to ensure that representative samples were taken. The samples were analysed at the Norwegian College of Veterinary Medicine using HPLC (High Performance Liquid Chromatography).

The half-life for DSP was calculated using the following formula:

T_{1/2}=t·log 0.5/(log N−log N₀)

T_{1/2}=half life
N₀=DSP concentration at the start
N=DSP concentration after time t, and
t=time, duration of the test

### Results of the Feed Test

1. Feeding with Plant Material

Plant material such as clover, lettuce, dandelion, spinach etc. was boiled in order to give a positive result. Crushing was done using a handmixer. Half-life: 14-15 days at 10° C. over a period of 2 weeks. In the third week the half-life was considerably longer. This may indicate that the feed lacks essential compounds.

2. Feeding with Whey and Milk

Whey and milk were used as feed without pre-treatment. Half-life: about 20 days. Small particles (less than 10 μm) of the whey/milk came out whole in the faeces. The nutrients in them were thus not accessible in the bivalve's digestive system.

3. Feeding with Fish Meat

Fish meat (herring and mackerel), crushed to a suitable particle size gave a half-life of 13-19 days during the duration of the test. This may indicate that essential compounds are present in the feed. Problems of pseudofaeces were observed when more than 1 g of feed per kg of bivalves per day was used.

4. Feeding with Bioprotein Containing Phospholipids

Feeding with just BioProtein® (3 grams per kg of bivalves per day) at about 10° C.

### Result:

| | |
|---|---|
| Start 0 hours: | 1160 μg DSP per kg |
| After 7 days: | 632 μg DSP per kg |
| After 14 days: | 439 μg DSP per kg |
| After 26 days: | 320 μg DSP per kg |

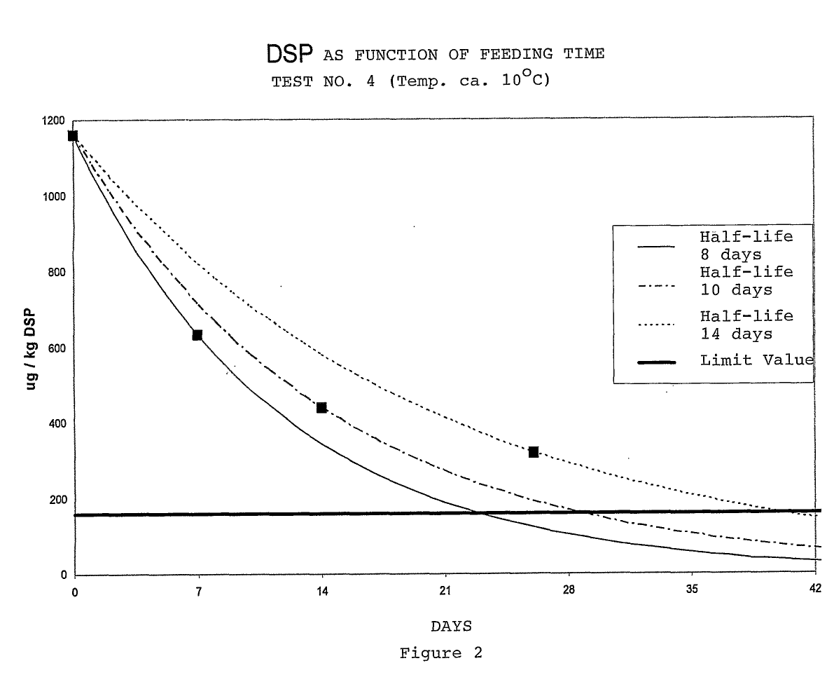

The calculated half-life for the DSP content was 8 days when calculated after 7 days. There were no pseudofaeces on feeding with substantially more than 1 gram per kg of bivalves per day. The half-life calculated over 14 days was 10 days and over 26 days was 14 days (FIG. 2). This is indicative of a lack of essential compounds in the feed.

5. Feeding with Bioprotein and Emulsified Oil

Test No. 4 was repeated using different cooking oils, fish oils and vitamin C separately in addition to bioprotein. The water temperature was 7.2° C., which results in a longer half-life. However, the half-life was “stable” when cooking oil and fish oil were used. Vitamin C had no effect. The best results were obtained when using cod liver oil emulsified in soft roe. The feed composition in this test was:

| | |
|---|---|
| Basic BioProtein ® (Norferm AS) | 2.5 g (83%) |
| Cod liver oil | 0.1 g (3.3%) |
| Soft roe | 0.1 g (3.3%) |
| Modified starch | 0.3 g (10.4%) |
| Vitamin C | trace amounts |

### Result:

| | |
|---|---|
| Start 0 hours: | 412 μg DSP per kg |
| After 10 days: | 259 μg DSP per kg |
| After 20 days: | 141 μg DSP per kg |
| After 29 days: | 81 μg DSP per kg |

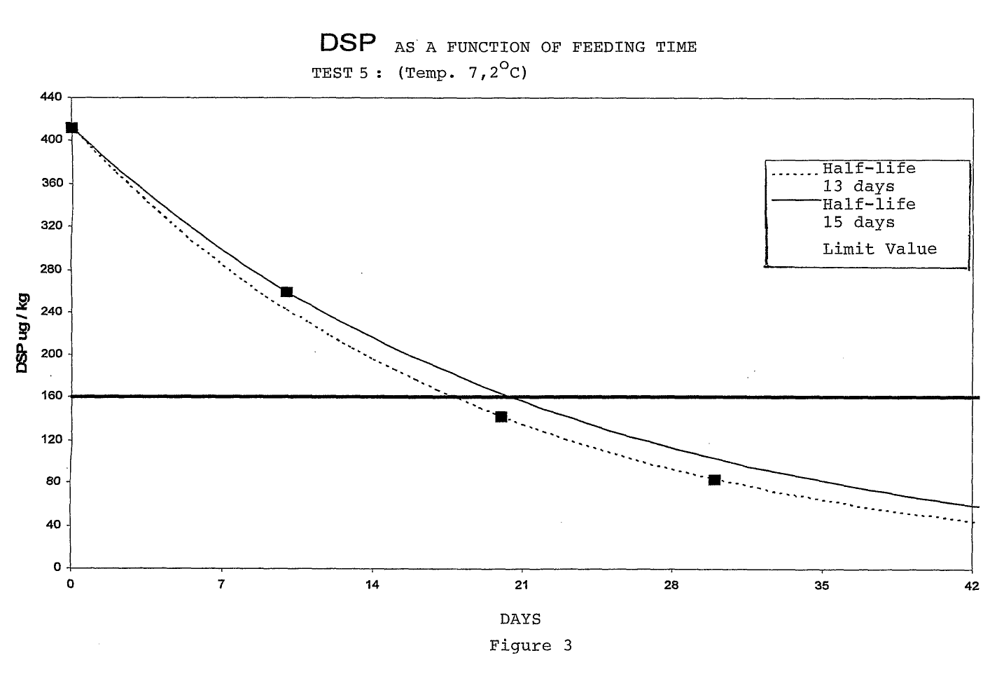

The calculated half-life for the DSP content was 13 to 15 days at 7.2 degrees Celsius during the duration of the test (4 weeks) (FIG. 3)

The calculated half-life for the DSP content was 15 days calculated after a detoxification period of 10 days. There were no pseudofaeces on feeding with substantially more than 1 gram per kg of bivalves per day. The half-life calculated over a period of 20 days and 29 days was 13.

Tests 4 and 5 show that bivalves fed with a feed composition containing an emulsifier reduces the half-life for the removal of DSP-toxin to 8-15 days as opposed to algal feed where the half-life is at best from 18-26 days.

### 6. Tests on an Industrial Scale

Tests shows that the results are reproducible on an industrial scale

### 7. Effect of Temperature and Salinity

Tests show that temperature and salinity of the water affect the effect of the feed.

### Results of Washing and Rinsing Tests

8. Batchwise Washing with a High Quantity of Emulsifier and Long Rinsing Time

The test was carried out in a vessel as described above at a temperature of 6-8 degrees Celsius.

### Washing Phase:

Emulsifier in the form of lecithin (4 grams per kg of bivalves) was added in a batch to a recycling system which ran for 8 hours.

### Rinsing Phase:

The washing phase was followed by a rinsing phase which lasted 12 hours. That is to say that the bivalves were kept for 12 hours in through-flowing purified seawater. The treatment was repeated several times.

Samples were taken immediately prior to the start of washing and after 18 hours.

### Result:

| | |
|---|---|
| Start 0 hours: | 63 μg DSP per kg |
| After 18 hours: | 31 μg DSP per kg |

The calculated half-life after 18 hours was 18 hours.

9. Batchwise Washing with a High Quantity of Emulsifier and Short Rinsing Time

The test was carried out in the same way as Test 8, but the rinsing time was reduced to 30 minutes between each wash.

### Result:

| | |
|---|---|
| Start 0 hours: | 1734 μg DSP per kg |
| After 18 hours: | 1475 μg DSP per kg |
| After 30 hours: | 1401 μg DSP per kg |

The calculated half-life after 18 hours was about 3 days (77 hours) and after 30 hours about 4 days (98 hours).

10. Batchwise Washing with a Low Quantity of Emulsifier and Short Rinsing Time

The test was carried out in the same way as Test 9, but the amount of lecithin was reduced to 2 g of lecithin (batch) per kg of bivalves.

### Result:

| | |
|---|---|
| Start 0 hours: | 620 μg DSP per kg |
| After 5.5 hours: | 569 μg DSP per kg |
| After 12 hours: | 547 μg DSP per kg |

The calculated half-life after 5.5 hours was about 44 hours, i.e., 2 days, whilst the calculated half-life after 12 hours was 60 hours, i.e., about 3 days.

11. Continuous Washing with a Low Quantity of Emulsifier and Long Rinsing Time

This test was carried out by adding via a dosing pump 2 g of lecithin per kg of bivalves over a time period of 4 hours. Treatment time 8 hours and rinsing time 12 hours.

### Result:

| | |
|---|---|
| Start 0 hours: | 687 μg DSP per kg |
| After 10 hours: | 497 μg DSP per kg |

The calculated half-life after 10 hours was 21 hours, i.e., just less than one day.

The present invention shows that the half-life for detoxification of bivalves that have been subjected to the washing and rinsing processes according to the invention is reduced to a few days compared with a half-life of 18-26 days for detoxification of bivalves that have not been subjected to the washing and rinsing processes according to the invention.

1. A method for detoxifying bivalves and other shellfish, wherein the shellfish are supplied with an emulsifier.

2. The method according to claim 1, wherein the shellfish are fed with a composition containing standard feed components and an emulsifier.

3. The method according to claim 1, wherein the bivalves and shellfish are washed and rinsed using a medium containing an emulsifier.

4. The method according to claim 1, wherein the emulsifier is a phospholipid.

5. The method according to claim 1, wherein the emulsifier is a lecithin.

6. Use of an emulsifier for the detoxification of bivalves and other shellfish.

7. The use according to claim 6, wherein the emuslifier is a phospholipid.

8. The use according to claim 6, wherein the emuslifier is a lecithen.

9. The use according to claim 6, wherein the toxin is lipophilic, in particular DSP.
